(19) United States
(12) Patent Application Publication
(10) Pub. No.: US 2007/0166709 A1
(43) Pub. Date: Jul. 19, 2007

(54) Immuno-pcr method

(75) Inventors: David Thomas McCreavy, Liverpool (GB); William Ducan Fraser, Liverpool (GB); James Anthony Gallagher, Liverpool (GB)

Correspondence Address: CROWELL & MORING LLP;INTELLECTUAL PROPERTY GROUP, P.O. BOX 14300, WASHINGTON, DC, 20044-4300, US

(73) Assignee: THE UNIVERSITY OF LIVERPOOL, LIVERPOOL (GB)

(21) Appl. No.: 10/545,662

(22) PCT Filed: Jan. 21, 2004
(86) PCT No.: PCT/GB04/00193

§ 371 Date: Aug. 16, 2006

(30) Foreign Application Priority Data
Feb. 15, 2003 (GB) 0303497.0

(51) Int. Cl. C12Q 1/68; C12P 19/34

(52) U.S. Cl. 435/6; 435/91.2

(57) ABSTRACT

An immunoassay utilizing the polymerase chain reaction to detect biological molecules in a sample. In one embodiment, the method employs a plurality of nucleic acid templates with varying annealing temperatures when annealed to a detectable probe.

The invention relates to an immunoassay which utilises the polymerase chain reaction to detect a plurality of biological molecules in a sample.

A diagnostic test has to have a very high degree of sensitivity and specificity if it is to have value in predicting the early on set of disease. There are a number of molecules present within serum, for example, interleukins and parathyroid hormone related protein, which are potential markers of cancer and other pathological conditions. Currently these are only measurable during the late stages of the disease process when they are overexpressed by tumours. Under normal conditions the proteins are present at concentrations <0.1 pM. Moreover, the early detection of pathogenic organisms in an infection can be critical to whether or not an infected animal survives the infection. This is particularly the case in diseases such as bacterial meningitis and septicemia caused, for example by Staphyloccocus aureaus. The earlier these molecules can be measured during the disease process the better the prognosis. However, early detection means that the molecules are at low concentrations and the signaling/quantitation systems of current immunoassays, using enzymes and chemiluminescence does not provide sufficient sensitivity to measure at these low levels.

In addition, there are many clinical scenarios where the simultaneous measurement of more than one biological molecule in a sample would be of significant diagnostic and therapeutic benefit. Some obvious examples relate to the investigation of endocrine abnormalities where the interpretation of a single result would be significantly affected by another result.

In thyroid disease a combination of thyroid stimulating hormone (TSH) with an index of thyroid hormone status (total or free thyroid hormone) is essential to make an accurate diagnosis in many cases. In investigation of calcium disorders a combination of Parathyroid Hormone (PTH) and Parathyroid Hormone Related Protein (PTHrP) would differentiate the two major causes of hypercalcaemia and elucidate the presence of dual pathology which is often overlooked. When problems of sexual dysfunction and infertility are investigated the pituitary hormones luteinising hormone (LH) and follicle stimulating hormone (FSH) are measured with oestradiol and/or testosterone included in the hormone profile. Another major diagnostic area is the screening for Down's syndrome where a combination of hormone measurements (Human Chorionic Gonadotrophin (HCG), alpha feto-protein (αFP) and maternal age is used to assess risk of the fetus having Down's syndrome. It is clear that the development of an assay system able to simultaneously measure a number of analytes would have numerous diagnostic applications.

Immuno-PCR is a method which combines both antibody technology and the polymerase chain reaction or other means to detect a nucleic acid probe conjugated to the antibody. In essence, immuno-PCR utilises an antibody to which a nucleic acid probe has been conjugated. The conjugate binds an antigen to be detected via the antibody part and non-bound conjugate is washed from the sample. The bound antibody is then detected by a PCR reaction which amplifies the nucleic acid part of the conjugate. The assay provides a sensitive and specific test for a biological molecule with specificity being provided by the antibody and sensitivity by the PCR detection of the nucleic acid conjugated to the antibody. We disclose a variation on the immuno-PCR method, referred to as Multiple Analyte Quantitation through Single Stranded Extension (MAQSSE), which is described in WO 03/048388, the contents of which are herewith incorporated by reference.

Briefly, WO 03/048388 discloses the use of a single stranded DNA reporter molecule as a means of detecting a ligand/DNA conjugate which is bound to a target molecule the detection of which is desired. This method is referred to as Multiple Analyte Quantitation through Single Stranded Extension (MAQSSE).

An oligonucleotide is conjugated to a ligand which has specificity for a biological molecule. The ligand/DNA conjugate is incubated with a sample and binds a target biological molecule. A single stranded DNA template (ssDNA) of defined length is then added to the reaction and anneals to the bound oligonucleotide. A DNA polymerase and deoxynucleotide triphosphates are added and the reaction incubated to elongate the primed oligonucleotide strand to produce a dsDNA. A nuclease specific for ssDNA is added to the reaction to degrade the background ssDNA template, resulting in no detectable background template. To generate a measureable signal, PCR is undertaken to amplify the double stranded DNA followed by detection.

A variation of this method is also disclosed in WO 03/048388 which simplifies the method and avoids the addition of an exogenous nuclease to digest ssDNA which is not bound to the conjugate/biological molecule complex. The variation comprises a ligand:oligonucleotide conjugate wherein the oligonucleotide has a bipartite sequence structure. The conjugate thus formed is contacted with a test sample which potentially includes a biological molecule to which the ligand binds. The bound conjugate is then incubated with the ssDNA template. The bipartite oligonucleotide is able to anneal over part of its length to a region of the ssDNA. The annealed bipartite oligonucleotide is extended by DNA polymerase to form a double standed DNA. An excess of oligonucleotide primer is added to the reaction mix, the sequence of which is able to anneal to that part of the bipartite oligonucleotide which is not annealed to the ssDNA template. A polymerase chain reaction is then conducted. Only the ssDNA which has annealed to the bipartite oligonucleotide is capable of being subsequently amplified therefore the assay provides a highly specific and sensitive means to monitor the presence of biological molecules. Preferably the bipartite oligonucleotide comprises a palindromic sequence. The use of primers which are palindromes of one another (i.e. a palindromic sequence is a sequence which has the same sequence when read in a 5′-3′ direction as when read in a 3′-5′ direction. For example a palindrome of the sequence: 5′ GGGCAAACGGG 3′ is 3′ GGGCAAACGGG 5′) decreases background noise and allows accurate PCR conditions to be established thereby providing an reliable test.

We have further adapted the above method to provide a means to simultaneously measure a plurality of biological agents in a single sample in a single reaction mixture. The invention utilises melt curve analysis to facilitate the detection.

The relative thermostability or Tₘ of a polynucleotide depends primarily upon its guanine (G) and cytosine (C) content. Tₘ is also influenced, although to a lesser extent, by the length of the nucleotide sequence and the distribution of G+C. For example so called GC clamps at the end of a polynucleotide sequence can increase Tₘ without increasing the overall G+C content. The detection and comparison of DNA sequences based on melting temperature is known to those skilled in the art. These methods can generally be categorised into three different types. Firstly, measuring a signal from a specifically bound reporter molecule which indicates the presence or absence of a polynucleotide sequence. Secondly, and as outlined in JP 3-147796A, a melt curve is generated as an indicator of the presence or absence of a polynucleotide sequence. Thirdly, a method whereby melt curves are compared, each melt curve being characteristic of a distinct polynucleotide sequence.

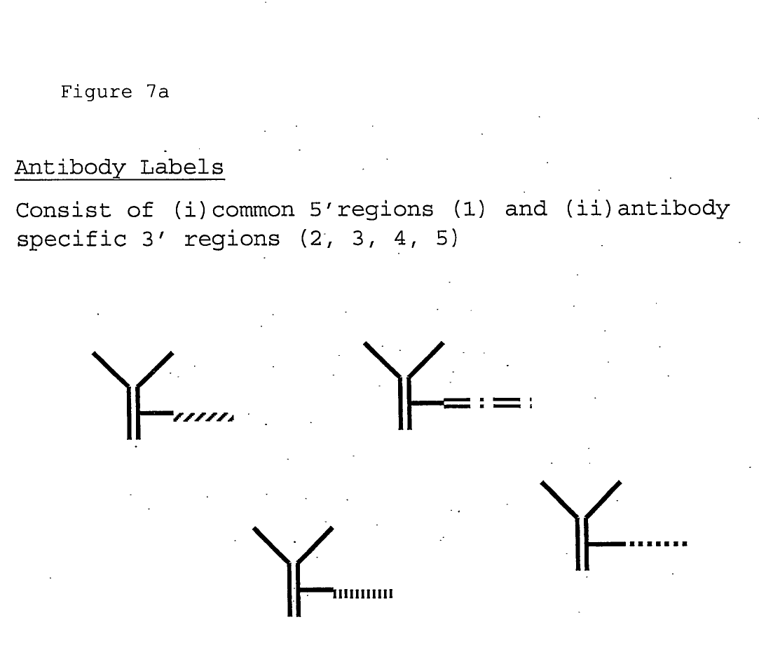

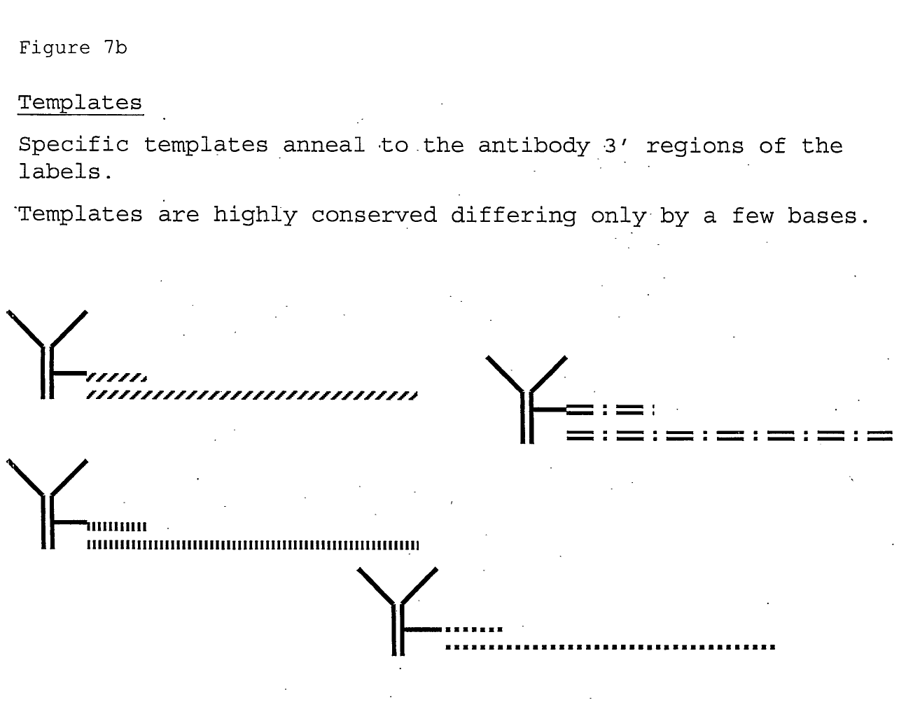

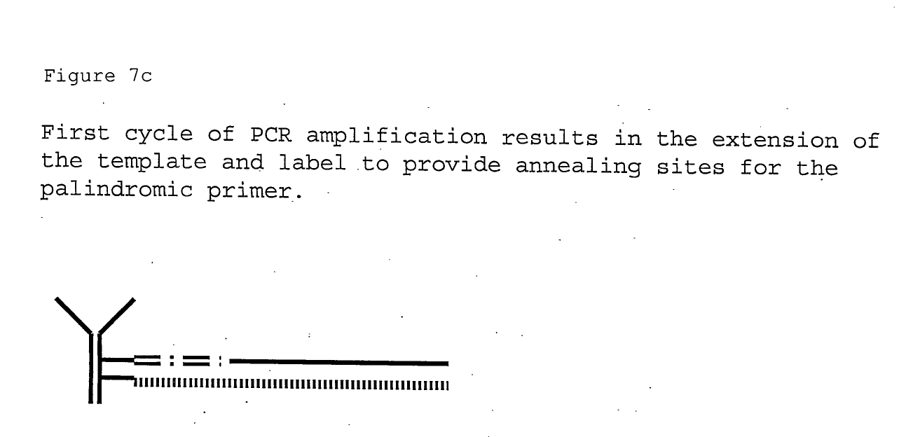

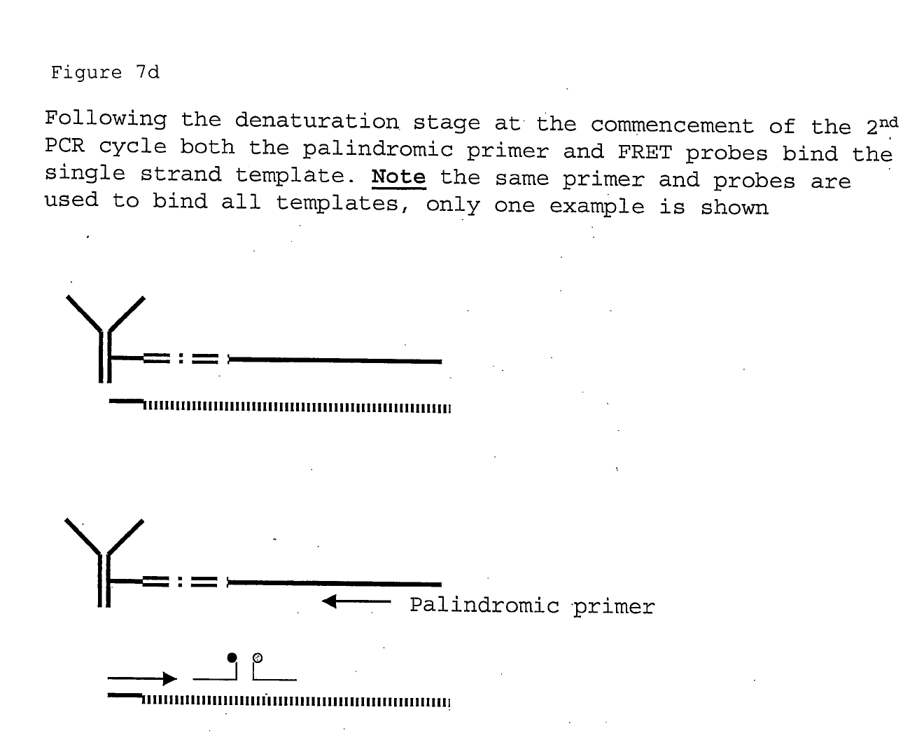

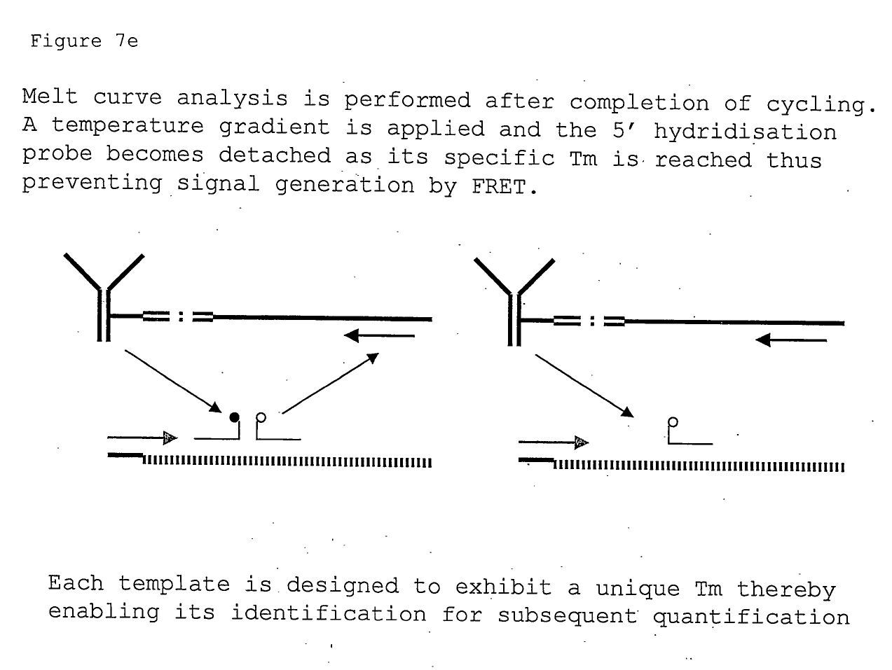

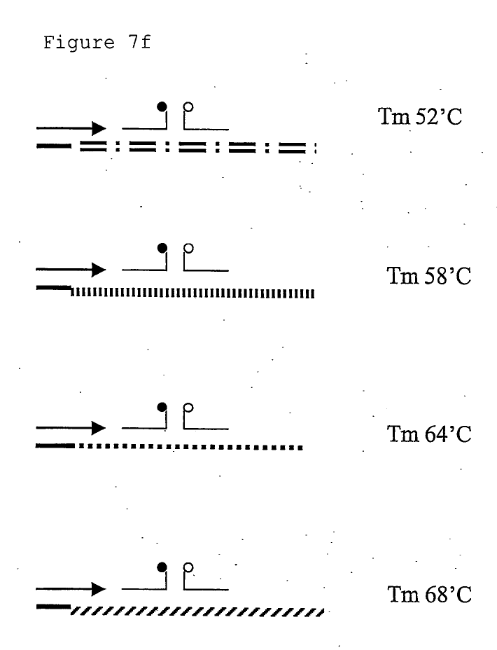

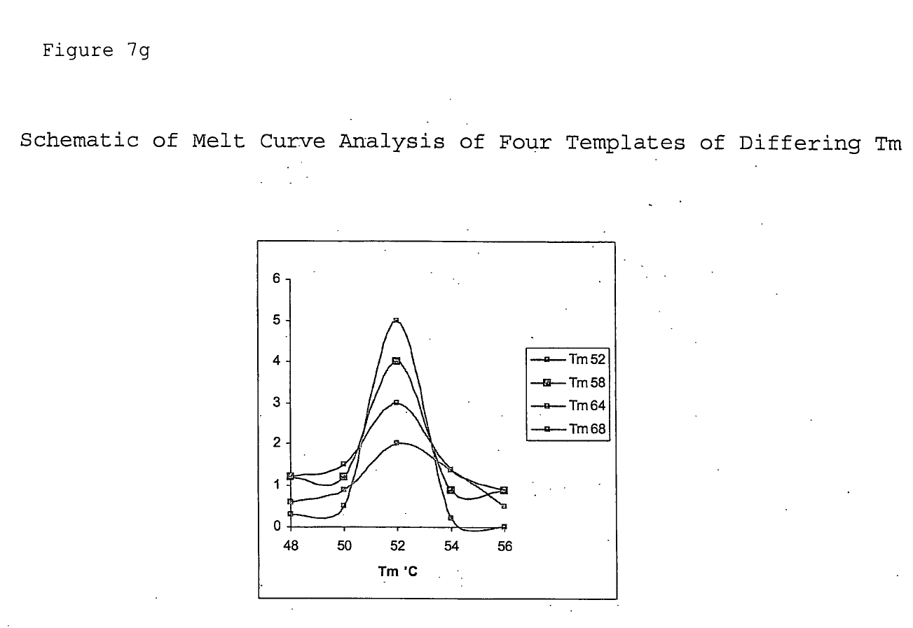

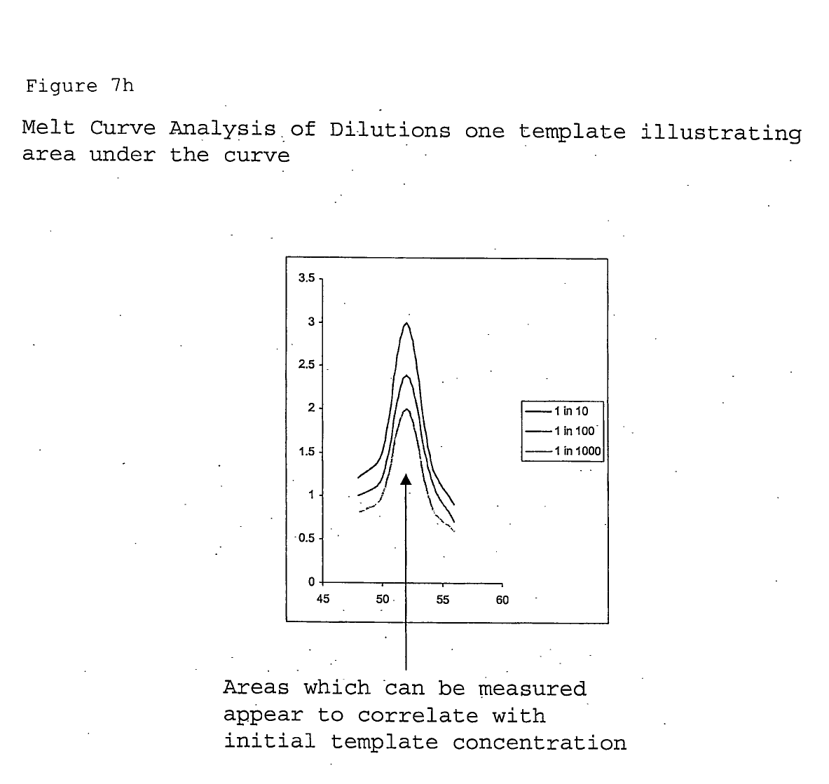

We have developed a series of ssDNA reporter molecules of differing Tm which allows a plurality of biological molecules to be detected in a single sample. The method is schematically illustrated in FIGS. 7a-h. By applying melt curve analysis to identify specific amplified PCR labels through differences in annealing of a common pair of probes to the templates. Each template/probe interaction will have a specific Tm and through melt curve analysis the area under the curve can be measured to determine the concentration of the template.

According to an aspect of the invention there is provided an immuno polymerase chain reaction method to detect a plurality of biological molecules in an isolated sample comprising: providing a sample to be tested and a preparation including a plurality of ligands wherein said ligands are conjugated or associated, directly or indirectly, with an oligonucleotide molecule(s); a plurality of nucleic acid template molecules adapted to anneal to said oligonucleotide molecule(s) and at least one detectable probe molecule adapted to anneal to said template molecule wherein said template molecule(s) comprise a nucleic acid sequence which is modified by addition, deletion or substitution of at least one nucleic acid base wherein said modifications provide a plurality of templates which vary from one another such that the annealing temperatures of said templates to at least one detectable probe molecule is varied.

According to an aspect of the invention there is provided an immuno polymerase chain reaction method to detect a plurality of biological molecules in a sample wherein said method comprises the steps of:
i) providing a preparation comprising;
   a) an assay sample to be tested; and
   b) a plurality of ligands specific for at least one biological molecule in said assay sample wherein each ligand is coupled to an oligonucleotide;
ii) incubating said preparation under conditions which allow the binding of said ligands to said biological molecules to form a complex;
iii) adding to the first preparation a second preparation comprising;
   c) a plurality of single stranded nucleic acid templates wherein said template molecules comprise a nucleic acid sequence which is modified by addition, deletion or substitution of at least one nucleic acid base wherein said modifications provide a plurality of templates which vary from one another such that the annealing temperatures of said templates to at least one complementary detectable probe molecule is varied and which at least part of said template is adapted to anneal to the oligonucleotide of (b); and
   d) at least one oligonucleotide primer and polymerase chain reaction reagents;
iv) conducting a polymerase chain reaction to amplify said template; and
v) conducting melt curve analysis during said reaction to determine the presence of said biological molecule.

In a preferred method of the invention there is provided an assay sample selected from the group consisting of a sample of blood; serum; semen; lymph fluid; cerebrospinal fluid; tears; saliva; urine; sweat. The assay sample may be an environmental sample, for example soil or water.

More preferably said method is for use as a diagnostic tool in clinical situations where the simultaneous measurement of more than one analyte would be of significant diagnostic and therapeutic benefit, for example in the investigation of endocrine abnormalities.

In a further preferred method of the invention said ligands are polypeptides.

In a yet further preferred method of the invention said polypeptides are antibodies, or at least the effective binding part thereof. Preferably said antibodies are monoclonal antibodies, or at least the Fab fragment of said monoclonal antibody.

In a further preferred method of the invention said ligands are receptors.

In a further preferred method of the invention said biological molecules are associated with a disease condition, for example cancer (e.g. a tumour rejection antigen) Tumour rejection antigens are known in the art, for example and not by way of limitation, the MAGE, BAGE, GAGE and DAGE families of tumour rejection antigens, see Schulz et al Proc Natl Acad Sci USA, 1991, 88, pp 991-993.

In a further preferred method of the invention said biological molecule is an antigenic polypeptide expressed by a pathogen. For example a viral, bacterial or parasitic pathogen.

It will be clear to one skilled in the art that the ligands can be antibodies which are specific for a biological molecule which may be present in said assay sample. Alternatively the biological molecule may be labelled with the oligonucleotide and the antibody specific for said biological molecule detected in the assay sample.

In a further preferred method of the invention each single stranded nucleic acid templates comprise a conserved nucleic acid sequence each of which is modified by addition, deletion or substitution of at least one nucleic acid base wherein said modifications provide a plurality of probes which vary from one another such that the annealing temperatures of said probes to a complementary sequence is varied. It is well established in the art that methods are available to manufacture nucleic acids of defined sequence and base composition.

Preferably still the annealing temperatures of said templates for said detectable probe is in a range of between 40 to 80° C. More preferably still, said annealing temperatures are in the range of between 50 to 70° C.

It will be apparent that the skilled person can readily design probes of defined nucleotide sequence which vary from one another in annealing temperature for a given template under defined hybridisation conditions, see Sambrook et al Molecular Cloning, A laboratory Manual, 1989.

In a further preferred method of the invention said detectable probe is a fluorescently labelled probe, preferably a pair of fluorescently labelled probes and detection is via fluorescence resonance energy transfer.

FRET is a distance-dependent interaction between the electronic excited states of two dye molecules in which excitation is transferred from a donor molecule to an acceptor or quencher molecule whilst returning the donor molecule to its lower energy level or ground state without fluorescence emission.

In a further preferred method of the invention said oligonucleotide primer comprises a palindromic sequence.

A palindromic sequence is a sequence which has the same sequence when read in a 5′-3′ direction as when read in a 3′-5′ direction. For example a palindrome of the sequence: 5′ GGGCAAACGGG 3′ is 3′ GGGCAAACGGG 5′. The use of a single palindromic primer to amplify the ssDNA allows accurate PCR conditions to be established thereby providing a reliable test.

According to a further aspect of the invention there is provided a preparation comprising a plurality of single stranded nucleic acid template molecules wherein said molecules comprise a nucleic acid sequence which is modified by addition, deletion or substitution of at least one nucleic acid base wherein said modifications provide a plurality of templates which have altered annealing temperatures for at least one complementary single stranded nucleic acid molecule capable of annealing to said template.

In a preferred embodiment of the invention said composition further includes at least one complementary detectable probe molecule. Preferably said probe includes at least one fluorescent label.

According to a further aspect of the invention there is provided a kit comprising: a plurality of ligands; oligonucleotide molecules; a plurality of nucleic acid templates; and a detectable probe molecule(s). Optionally said kit includes polymerase chain reaction reagents.

An embodiment of the invention will know be described by example only and with reference to the following figures:

FIGS. 1, 2, 3, 4, 5, and 6 illustrates oligonucleotide probes used in the method of the invention, and

FIGS. 7a-h is a schematic representation of the use of melt curve analysis to detect a plurality of biological molecules;

### MATERIALS AND METHODS

(i) Label Design Procedure

pcDNA3.1v5histopo.seq (Invitrogen) was loaded into Oligo6,

Two primer pairs (in bold) were chosen see FIG. 1

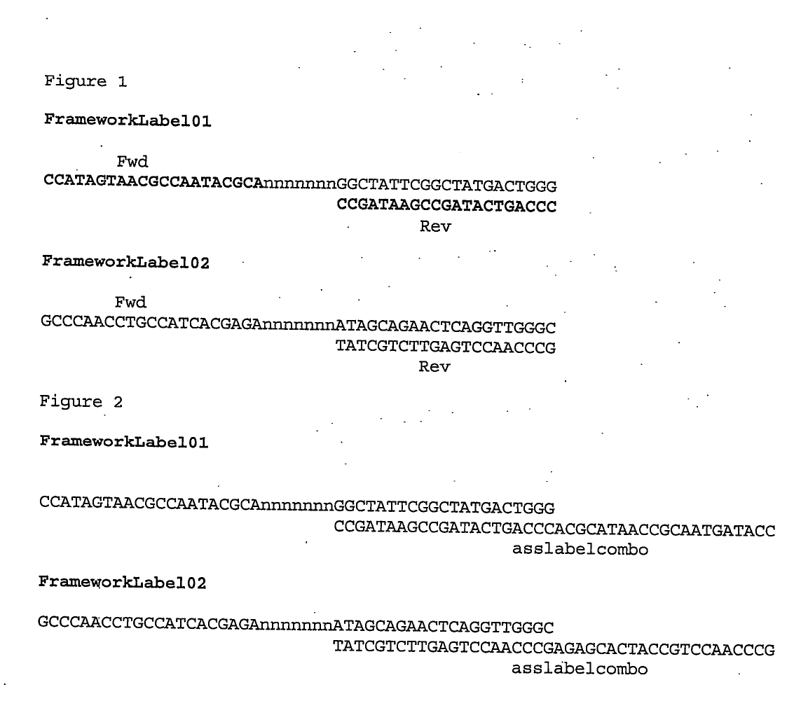

The forward sequences were appended to the 5′ region of the Rev sequences, to create palindromic binding sites see FIG. 2

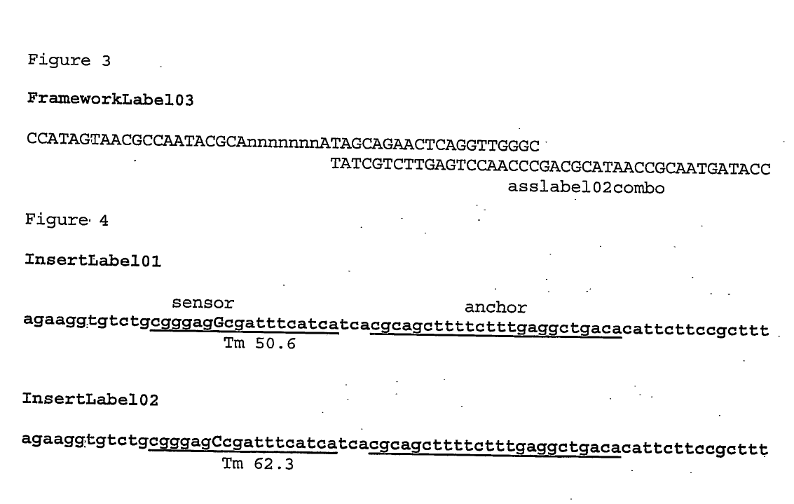

FrameworkLabel03 was produced by replacing the appended forward sequence of FrameworkLabel02 with that of FrameworkLabel01, see FIG. 3

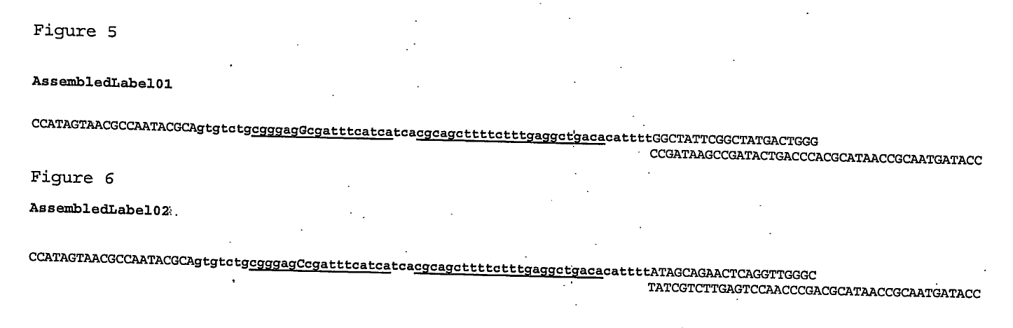

Insert labels were designed to replace the nnnnn region and consisted of two binding sequences one termed the sensor and the second the anchor. MeltCalc software was used to design two sensor sequences differing in respect to one base G-C whose Tm differed by over 10°C. The anchor primer was labelled at the 3′ with fluorescein. The anchor was designed to bind from four bases downstream and be labelled with LC 640 at its 5′ end thereby facilitating FRET when both were simultaneously bound, see FIG. 4. nnnnn sequences in the Frameworklabel01 and 03 were replaced by InsertLabel01 see FIG. 5 and InsertLabel02 respectively see FIG. 6

(ii)Conjugation of Labels to Monoclonal Antibody

The asslabel01combo and asslabel02 combo are synthesized with an amino linker at their 5′ termini to facilitate conjugation to reduced detector antibodies using the heterobifunctional cross linking reagent sulfo succinimidyl 4-(N-maleimidomethyl) cyclohexane-1-carboxylate.

Antibody Reduction

1 ml of 1 mg/ml anti-TSH monoclonal antibody was desalted/buffer exchange using PD10 columns pre equilibrated with 100 mM sodium phosphate 5 mM EDTA pH6.0. Positive fractions were pooled and concentrated (Sartorius 30 kDa MWCO) to 1 ml and added to 1 vial Perbio 2Mercaptoethyamine and incubated at 37′C. for 90 mins. The solution was concentrated (Sartorius 30 kDa MWCO) to 500 ul and applied to a PD10 column pre-equil with PBS 5 nM EDTA pH7.0. Protein containing fractions were pooled and concentrated to 100 ul.

Asslabelcombo S-SMCC treatment

250 μl 1 M sodium phosphate pH8.0 was added to 115 μl water followed by 80 μl of asslabelcombo(2 mg/ml in water). 55 μl of 1 mg/ml S-SMCC was added and mixed thoroughly. The solution is vortexed and heated to 37′C. for 20 mins. The solution was applied to a PD10 column pre-equil with PBS pH7.0. Fractions containing the label were pooled and concentrated to 100 μl using a Sartorius 3 kDa MWCO column

Conjugation

The antibody and asslabelcombo solutions were pooled and incubated with agitation in the dark at room temp for 2 hrs and overnight at 4′C.

Protein G Purification

The 200 μl was applied to a TBS pH8.5 pre-equilibrated Protein G column and eluted with 100 mM glycine pH2.5. 1 ml fractions were collected and assayed for protein using a BCA kit (Sigma) and oligonulceotide using OliGREEN. Positive fractions were pooled and concentrated to 200 μl, aliquoted and stored a −20′C.

(iii) Coating of HyBaid Polycarbonate PCR Plates

Binding Buffer

42 mg NaHCO3 dissolved in 100 ml deionised water, pH adjusted to 8.5

TBS/Tween

1.5 g Trisma dissolved in 500 ml of deionised water, pH adjusted to 7.4. 1.450 g of NaCl added and 250 μl of Tween-20

TBS-BLA

2 g polypep, 10 g beta-lactose, 100 mg/ml salmon sperm DNA (from 10 mg/ml frozen stock) added to 100 ml TBS/Tween

Procedure

50 μl of a solution containing 5 μg/ml of anti-TSH and anti-HCG monoclonal antibodies in binding buffer was added to wells and incubated overnight at room temperature. Wells were washed three times with fresh TBS/Tween, using a BioHit plate washer. 150 μl of TBS-BLA was added to wells and incubated for 1 hr at room temperature. The wells were aspirated using the plate washer, drained and inverted to remove excess solution. The plates were air dried overnight.

(iv) Assay Procedure

25 μl of TSH and HCG diluted in TBS, 2%pp, 2%BSA, 0.1% Tween was added to Hybaid coated wells and incubated for 1 hour at room temperature with agitation. The plate was washed 3 times with TBS-Tween using a BioHit plate washer. 25 μl of anti-TSH-asslabelcombo1 and anti-HCG-asslabelcombo2 diluted in TBS, 2% pp, 2% BSA, 0.1% Tween was added to the wells and incubated for 1 hour at room temperature with agitation. The plate was washed 3 times with TBS-Tween and 3 times with de-ionised water. 25 μl of 10 mM Tris pH8 was added to the wells which were then heated to 95′C. for 5 minutes.

(v) PCR amplification and Melt Curve Analysis

PCR will be undertaken using the Roche Lightcycler. A reaction mix containing the following will be prepared.

| | μl |
|---|---|
| Sterile water | 9.6 |
| MgCl₂ | 2.4 |
| Palindromic primer final conc 0.2 μM | 2.0 |
| FrameworkLabel01 final conc 0.2 μM | 1.0 |
| FrameworkLabel03 final conc 0.2 μM | 1.0 |
| Sensor/anchor hybridization probes | 2.0 |

Mixed and pipetted into a precooled capillary. 2 μl of denatured complex were added, the capillary capped and the reaction commenced (see Table 1)

(vi) Melt Curve Analysis

On completion of PCR, data for each sample is converted into melting point profiles by calculating the negative first derivative of the fluorescence −dF/dt versus temperature. As the hybridization probes dissociate from the frameworklabels 01 and 03 at 50.6′C. and 62.3′C. respectively two curves are simultaneously displayed. The area under the curve which correlates with the concentration is calculated from a Gaussian Fit curve. In this case the area under the first curve centred around 50′C. correlates with the concentration TSH. The area under the second curve centred around 62′C. correlates with the concentration of HCG.

TABLE 1

| Program | | Hold Time | Slope | |
|---|---|---|---|---|
| Seg No | Temp ‘C. | sec | C’/sec | Acquisition Mode |
| | Denaturation | | | |
| 1 | 95 | 120 | 20 | None |
| | Cycling | | | |
| 1 | 95 | 3 | 20 | None |
| 2 | 62 | 4 | 20 | Single |
| 3 | 62 | 4 | 20 | None |
| 4 | 72 | 12 | 20 | None |
| | Melting | | | |
| 1 | 95 | 20 | 20 | None |
| 2 | 40 | 20 | 20 | None |
| 3 | 70 | | 0.1 | Continuous |

1. An immuno polymerase chain reaction method to detect a plurality of biological molecules in a sample wherein said method comprises the steps of i) providing a preparation comprising;
a) an assay sample to be tested; and
b) a plurality of ligands specific for at least one biological molecule in said assay sample wherein each ligand is coupled to an oligonucleotide;
ii) incubating said preparation under conditions which allow the binding of said ligands to said biological molecules to form a complex;
iii) adding to the first preparation a second preparation comprising;
a) a plurality of single stranded nucleic acid templates wherein said templates comprise a nucleic acid sequence which is modified by addition, deletion or substitution of at least one nucleic acid base wherein said modifications provide a plurality of templates which vary from one another such that the annealing temperatures of said templates to at least one detectable probe molecule is varied and which at least part of each of said templates is adapted to anneal to the oligonucleotide of (b); and
b) at least one oligonucleotide primer and polymerase chain reaction reagents;
iv) conducting -a polymerase chain reaction to amplify said template; and
v) conducting melt curve analysis during said reaction to determine the presence of said biological molecule.

2. A method according to claim 1 wherein said assay sample selected from the group consisting of blood; serum; semen; lymph fluid; cerebrospinal fluid; tears; saliva; urine; sweat.

3. A method according to claim 1 wherein said sample is an environmental sample.

4. A method according to claim 1 wherein said ligands are polypeptides.

5. A method according to claim 4 wherein said polypeptides are antibodies, or at least the effective binding part thereof.

6. A method according to claim 5 wherein said antibodies are monoclonal antibodies, or at least the Fab fragment of said monoclonal antibody.

7. A method according to claim 1 wherein said ligands are receptors.

8. A method according to claim 1 wherein said biological molecules are associated with a cancer.

9. A method according to claim 8 wherein molecule is a tumor rejection antigen.

10. A method according to claim 1 wherein said biological molecule is a polypeptide expressed by a pathogen.

11. A method according to claim 1 wherein each of said single stranded nucleic acid templates comprises a conserved nucleic acid sequence each of which is modified by addition, deletion or substitution of at least one nucleic acid base wherein said modifications provide a plurality of probes which vary from one another such that the annealing temperatures of said probes to a complementary sequence is varied.

12. A method according to claim 1 wherein said detectable probe anneals to said template in a range of between 40 to 80° C.

13. A method according to claim 12 wherein said annealing temperature is between 50 to 70° C.

14. A method according to claim 1 wherein said detectable probe is a fluorescently labeled probe.

15. A method according to claim 14 wherein said probe is a pair of fluorescently labelled probes and detection is via fluorescence resonance energy transfer.

16. A method according to claim 1 wherein said oligonucleotide primer comprises a palindromic sequence.

17. A preparation comprising a plurality of single stranded nucleic acid templates for use in the method according to claim 1, wherein said templates comprise a nucleic acid sequence which is modified by addition, deletion or substitution of at least one nucleic acid base wherein said modifications provide a plurality of templates which have altered annealing temperatures for at least one single stranded nucleic acid molecule capable of annealing to said templates.

18. A preparation according to claim 17 wherein said composition further includes at least one detectable probe molecule.

19. A preparation according to claim 18 wherein said probe includes at least one fluorescent label.

20. A kit comprising: a plurality of ligands; oligonucleotide molecules;
a plurality of single stranded nucleic acid templates wherein said templates comprise a nucleic acid sequence which is modified by addition, deletion or substitution of at least one nucleic acid base wherein said modifications provide a plurality of templates which have altered annealing temperatures for at least one single stranded nucleic acid molecule capable of annealing to said templates
and a detectable probe molecule(s).

21. A kit according to claim 20 wherein said kit includes polymerase chain reaction reagents.
